(19) United States
(12) Patent Application Publication
(10) Pub. No.: US 2025/0177718 A1
(43) Pub. Date: Jun. 5, 2025

(54) Device And Method For Sterile Connection Of Medical Tubes

(71) Applicant: Fresenius Kabi Deutschland GmbH, Bad Homburg (DE)

(72) Inventors: Tobias Kaiser, Frankfurt (DE); Michael Schäfer, Friedberg (DE); Lars Michel, Rosbach vor der Höhe (DE)

(21) Appl. No.: 18/840,673

(22) PCT Filed: Feb. 20, 2023
(86) PCT No.: PCT/EP2023/054132

§ 371 Date: Aug. 22, 2024

Related U.S. Application Data
(60) Provisional application No. 63/322,716, filed on Mar. 23, 2022.
(60) Provisional application No. 63/313,046, filed on Feb. 23, 2022.

(51) Int. Cl. A61M 39/18

(52) U.S. Cl. CPC A61M 39/18

(57) ABSTRACT

A sterile connection device includes two carriages configured to receive two tubes to be joined. A motor moves one carriage through a first portion of a path from an initial position in which the tubes are heated toward an intermediate position. At the time of this movement, the heated portion of each tube is at a temperature less than its melting temperature, with the movement severing the tubes and optionally moving heating elements of the carriages from a retracted condition toward a deployed condition. Movement of the carriage is paused at the intermediate position and the severed ends of the tubes are heated to a molten condition by the heating elements. Movement of the carriage then continues through a second portion of the path to a final position in which the heating elements retract (as necessary) to expose the severed ends of the tubes, which are joined together.

### RELATED APPLICATIONS

This application claims the benefit of and priority of U.S. Provisional Patent Application Ser. No. 63/313,046, filed Feb. 23, 2022, and U.S. Provisional Patent Application Ser. No. 63/322,716, filed Mar. 23, 2022, the contents of which are incorporated by reference herein.

### DESCRIPTION

### Technical Field

The invention relates to joinder of tubing. More particularly, the invention relates to systems and methods for sterilely joining tubing.

### Background

Fluid flow systems or assemblies that are pre-sterilized and/or pre-assembled are used in a wide variety of medical and non-medical applications. Medical applications may include, for example, administration of medical fluids to a patient for therapeutic and/or diagnostic purposes, blood and/or blood component or other cell collection or processing, dialysis, and other medical procedures. Non-medical applications for such systems or assemblies may include, for example, pharmaceutical manufacturing and cell processing. In the medical field in particular, such flow systems commonly employ one or more pre-filled containers or other sources of medical fluid or agent and an associated fluid flow circuit or system (sometimes called a tubing set) containing the necessary flow tubing, valves, flow controllers, process chambers, and the like to carry out the particular procedure, either alone or in cooperation with a reusable controller or other device. It is not unusual, for example, for a medical fluid flow system to include or be used in association with a container of a suitable drug, saline, anticoagulant, dextrose solution, sterile water, cell preservative, or the like, to name just a few examples.

Such a fluid flow system can, however, pose manufacturing or assembly challenges for different reasons. One reason can be that the pre-filled containers of medical liquid, powder, or other agent that is administered to the patient or otherwise employed in the medical fluid flow system, require different sterilization techniques than other portions of the fluid flow system. For example, empty plastic tubing, containers, flow control devices, and/or processing devices or chambers, which do not contain any substantial amount of liquid or other agent, may be sterilized with gamma or electron beam (e-beam) radiation or by exposure to a sterilizing gas, e.g., ethylene oxide. However, gas sterilization would be ineffective to sterilize an agent, such as a liquid, powder, or drug, contained in a sealed container, and exposing the agent to ionizing radiation may degrade or otherwise have a deleterious effect on the agent. Also, there may be situations where different portions of a sterile fluid flow system, even though suitable for the same sterilization process, are separately manufactured and sterilized for other reasons and then subsequently assembled in a sterile manner.

In addition, sterile connections often need to be made on-site, by the end user, e.g., at the location where the fluid flow systems are being used to treat patients or collect or process blood or blood components or biologic materials, or in other therapeutic or diagnostic procedures. As a result, a number of different approaches have been used in assembling sterile fluid flow systems. For example, one technique for manufacturing such systems employs the use of a sterile docking system, such as a device disclosed in U.S. Pat. No. 4,157,723, which is hereby incorporated herein by reference. As illustrated therein, the sterile docking system comprises a pair of mating members, each having a facing membrane. One of the mating members is connected to a pre-sterilized container of liquid, drug or other agent and the other mating member is attached to a pre-sterilized fluid flow system, which may include one or more empty containers. After the two members are joined, the docking system is exposed to radiant energy, causing the membranes to melt and form a sterile fluid pathway through the mating members. Fluid may then be transferred from the initial container into an empty container in the fluid flow system, and the flow path sealed and severed. The initial container and mating members are then discarded. While this works satisfactorily, it entails multiple manufacturing steps of transferring solution from one container to another in a sterile manner and the associated quality control procedures with such a step. It also requires the disposal of a portion of the product with increased product and waste cost.

According to an alternative approach, which is described in U.S. Pat. No. 4,978,446 (which is hereby incorporated herein by reference), sterilizing filters are used on the inlet flow line that couples a pre-sterilized liquid container or the like to a separately sterilized fluid flow tubing system. In this approach, medical personnel are required to manually join the fluid flow tubing system to the fluid container, such as by spiking the fluid container with a piercing member associated with the fluid flow system. In addition to the administrative requirements for individually ordering, storing, and prescribing solutions and disposable flow systems or sets, there is the added possibility of errors, such as by connection of a container of an incorrect liquid or other agent or an improper flow system to be used in association with the procedure.

There are also known devices commonly referred to as sterile tubing welders, with the device marketed by Terumo Medical Corporation as the TSCD-II sterile tubing welder being one example. That device uses a heated cutting element to slice and melt the ends of tubing, which are joined together after the cutting element is removed. Aspects of this device are disclosed in U.S. Patent Application Publication No. 2020/0047423, which is hereby incorporated herein by reference. One notable disadvantage of such devices is that they require the use of expensive cutting elements (each comprising a resistive circuit layer sandwiched between two copper layers) that are replaced after each splice.

Japanese Patent Application Publication No. H09-206383 A (which is hereby incorporated herein by reference) describes a device that replaces a disposable cutting element with reusable cutting elements. Rather than cutting entirely through a tube to be joined, the reusable cutting elements only partially cut through the walls of a pair of tubes to be joined. One tube holder of the device is moved in an arc with respect to another tube holder, which tears the tubes at their partially cut sections. At the end of the arc movement, the torn ends of the two tubes are aligned and pressed together to form a joint, resulting in a single, joined tube. As the cutting elements are never extended through or into the lumens of the tubes to be joined, the cutting elements cannot contaminate the fluid flow path defined by the lumen, such that the cutting elements may be reused, rather than being disposed of after a single use.

While more recent devices have improved upon older devices requiring disposable cutting elements, further improvements are possible. For example, certain devices require multiple heating technologies to join a pair of tubes, such as radio frequency energy being applied when sealing and severing the ends of a pair of tubes to be joined and radiant heat later being applied when joining the severed ends. Certain devices require multiple motors to move components of the devices in various directions. Devices that tear or pull apart uncut ends of a pair of tubes may heat the ends of the tubes to a high enough temperature that threads or fibers may be formed when the molten ends of the tubes are torn or pulled apart, with the threads or fibers “F” remaining when the tubes are subsequently joined, as shown in FIG. 1.

Sterile connection devices and methods according to the present disclosure address these and other disadvantages of known devices.

### SUMMARY

There are several aspects of the present subject matter which may be embodied separately or together in the devices, systems, and methods described and/or claimed below. These aspects may be employed alone or in combination with other aspects of the subject matter described herein, and the description of these aspects together is not intended to preclude the use of these aspects separately or the claiming of such aspects separately or in different combinations as set forth in the claims appended hereto or later amended.

The following summary is to acquaint the reader generally with various potential aspects of the present subject matter, and is non-limiting and non-exclusive with respect to the various possible aspects or combinations of aspects. Additional aspects and features may be found in the detailed description herein and/or in the accompanying figures.

In one aspect, a sterile connection device is provided with a first carriage, a second carriage, and a motor. The first carriage includes a lower jaw defining a first portion of a proximal slot configured to receive a portion of a proximal tube and a first portion of a distal slot configured to receive a portion of a distal tube. An upper jaw of the first carriage is configured to move between an open condition spaced away from the lower jaw and a closed condition positioned adjacent to the lower jaw. The second carriage is positioned laterally of the first carriage and includes a lower jaw defining a second portion of the proximal slot and a second portion of the distal slot. The second carriage has an upper jaw configured to move between an open condition spaced away from the lower jaw and a closed condition positioned adjacent to the lower jaw. The motor is configured to move the second carriage from an initial position to a final position. In the initial position, the first and second portions of the proximal slot are aligned and the first and second portions of the distal slot are aligned, with one of the portions of the proximal slot being aligned with one of the portions of the distal slot in the final position. The sterile connection device includes no motors other than the single motor used to move the second carriage.

In another aspect, a sterile connection device is provided with a first carriage, a second carriage, and a motor. The first carriage includes a lower jaw defining a first portion of a proximal slot configured to receive a portion of a proximal tube and a first portion of a distal slot configured to receive a portion of a distal tube. An upper jaw of the first carriage is configured to move between an open condition spaced away from the lower jaw and a closed condition positioned adjacent to the lower jaw. The second carriage is positioned laterally of the first carriage and includes a lower jaw defining a second portion of the proximal slot and a second portion of the distal slot. An upper jaw of the second carriage is configured to move between an open condition spaced away from the lower jaw and a closed condition positioned adjacent to the lower jaw. The motor is configured to move at least one of the carriages from an initial position to a final position. In the initial position, the first and second portions of the proximal slot are aligned and the first and second portions of the distal slot are aligned, with one of the portions of the proximal slot being aligned with one of the portions of the distal slot in the final position. Each carriage includes a waste-end heating element associated with one of the portions of the slots defined by that carriage and a joining heating element associated with the same portion or with the portion of the other slot defined by that carriage. Each waste-end heating element is configured to be operative in the initial position, with each joining heating element being configured to be operative when the movable carriage is in neither the initial position nor the final position.

In yet another aspect, a sterile connection device is provided with a first carriage, a second carriage, and a motor. The first carriage includes a lower jaw defining a first portion of a proximal slot configured to receive a portion of a proximal tube and a first portion of a distal slot configured to receive a portion of a distal tube. An upper jaw of the first carriage is configured to move between an open condition spaced away from the lower jaw and a closed condition positioned adjacent to the lower jaw. The second carriage is positioned laterally of the first carriage and includes a lower jaw defining a second portion of the proximal slot and a second portion of the distal slot. An upper jaw of the second carriage is configured to move between an open condition spaced away from the lower jaw and a closed condition positioned adjacent to the lower jaw. The motor is configured to move at least one of the carriages from an initial position to an intermediate position to a final position. In the initial position, the first and second portions of the proximal slot are aligned, the first and second portions of the distal slot are aligned, and a portion of each tube is heated. Moving the movable carriage from the initial position to the intermediate position causes the heated portion of each tube to be torn, defining a torn end. The heated portion of each tube is torn while it is at a first temperature that is less than its melting temperature. In the intermediate position, the torn end of each tube is heated to a second temperature greater than or equal to a melting temperature of the torn end. In the final position, one of the portions of the proximal slot is aligned with one of the portions of the distal slot so as to align and join the torn ends of the tubes.

In another aspect, a method of sterilely connecting two tubes includes heating a portion of each of two tubes and then severing each tube at the heated portion to define a severed end of each tube. The severed ends of the two tubes are moved into contact with each other and joined together, with the tubes being heated and joined without application of any type of energy other than radio frequency energy.

These and other aspects of the present subject matter are set forth in the following detailed description of the accompanying drawings.

### BRIEF DESCRIPTION OF THE DRAWINGS

FIG. 1 is a detail view of a portion of a joined tube formed by a sterile connection device configured according to conventional design;

FIG. 2 is a perspective view of a sterile connection device according to an aspect of the present disclosure, with a movable carriage thereof in an initial position;

FIG. 3 is a top plan view of the sterile connection device of FIG. 2, with a pair of tubes received by the movable carriage and a stationary carriage of the sterile connection device;

FIG. 4 is a top plan view of the sterile connection device and tubes of FIG. 3, with upper jaws of the carriages in a closed condition;

FIG. 5 is an elevation view of the side of the stationary carriage of the sterile connection device of FIGS. 2-4 facing the movable carriage, with a joining heating element thereof in a deployed condition;

FIG. 6 is a perspective view of the side of the stationary carriage of FIG. 5;

FIG. 7 is a perspective view of the side of the stationary carriage of FIGS. 5 and 6, with an upper electrode of the joining heating element thereof in a retracted condition;

FIG. 8 is a diagrammatic view of the carriages of the sterile connection device of FIGS. 2-4, with the movable carriage in its initial position;

FIG. 9 is a diagrammatic view of the carriages of the sterile connection device of FIGS. 2-4, with the movable carriage in an intermediate position;

FIG. 10 is a diagrammatic view of the carriages of the sterile connection device of FIGS. 2-4, with the movable carriage in a final position;

FIG. 11 is a top plan view of the sterile connection device of FIGS. 2-4, with the movable carriage in its final position, and with the upper jaws of the carriages in their closed condition;

FIG. 12 is a top plan view of the sterile connection device of FIGS. 2-4, with the movable carriage in its final position, and with the upper jaws of the carriages in an open condition;

FIG. 13 is a detail view of a joint of a joined tube formed by a sterile connection device configured according to the present disclosure;

FIG. 14 is a perspective view of another embodiment of a sterile connection device according to an aspect of the present disclosure, with a movable carriage thereof in an initial position;

FIG. 15 is a diagrammatic view of the carriages of the sterile connection device of FIG. 14, with the movable carriage in its initial position;

FIGS. 16 and 17 are diagrammatic views of the carriages of the sterile connection device of FIG. 14, with the movable carriage in intermediate positions; and

FIG. 18 is a diagrammatic view of the carriages of the sterile connection device of FIG. 14, with the movable carriage in a final position.

### DETAILED DESCRIPTION OF THE ILLUSTRATED EMBODIMENTS

The embodiments disclosed herein are for the purpose of providing a description of the present subject matter, and it is understood that the subject matter may be embodied in various other forms and combinations not shown in detail. Therefore, specific designs and features disclosed herein are not to be interpreted as limiting the subject matter as defined in the accompanying claims.

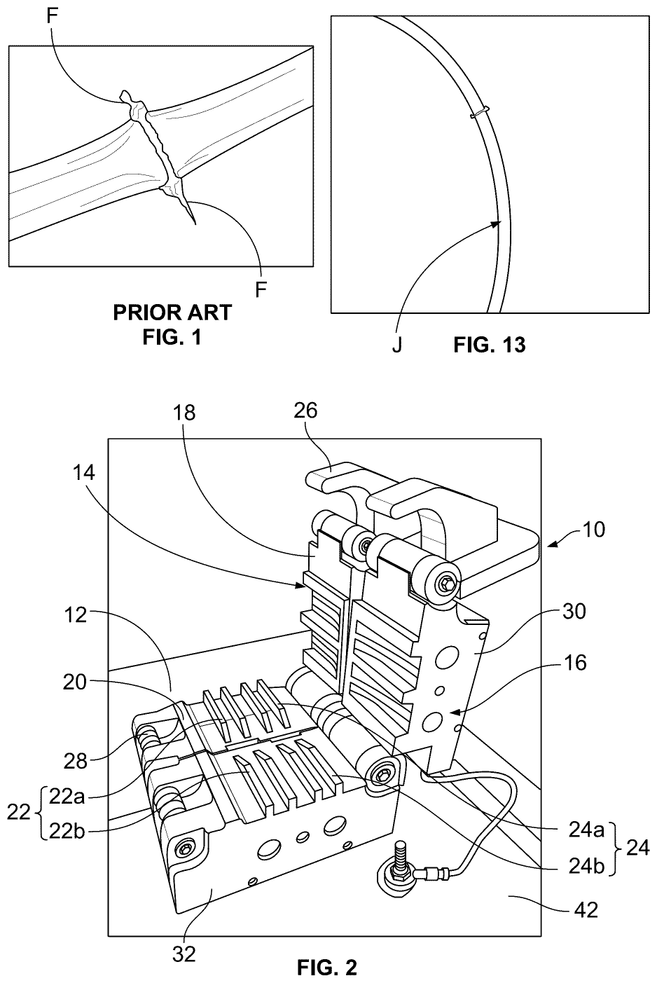

FIGS. 2-12 illustrate an exemplary embodiment of a sterile connection device 10 according to an aspect of the present disclosure. While sterile connection devices according to the present disclosure are particularly well-suited for sterile connection of tubes formed of plasticized polyvinyl chloride, it is within the scope of the present disclosure for the sterile connection device 10 to be used to sterilely connect tubes formed of other materials.

The illustrated sterile connection device 10 includes a housing 12 containing various components of the sterile connection device 10 (e.g., a controller, one or more power sources, thermocouples, etc.). The housing 12 may be variously configured without departing from the scope of the present disclosure, which may include the housing 12 having different portions being formed of, for example, a metallic material, a plastic material, or a combination of metallic and plastic materials.

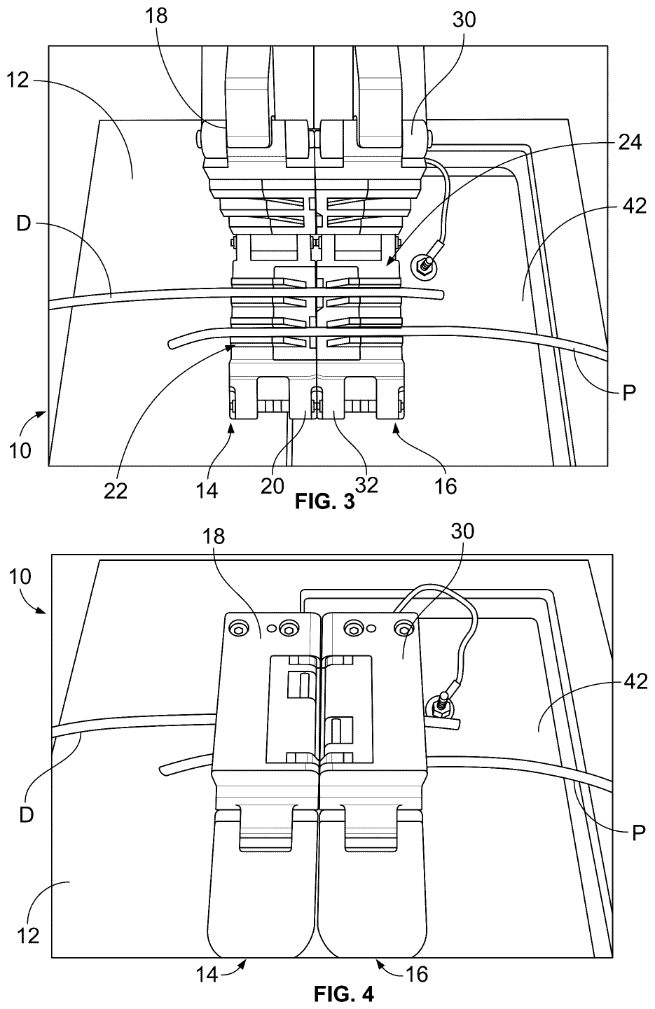

In the illustrated embodiment, first and second carriages 14 and 16 are associated with an upper surface or face of the housing 12, with the second carriage 16 being positioned laterally of the first carriage 14. At least a portion of at least one of the carriages 14, 16 is movable with respect to the housing 12 during a sterile connection procedure in which two tubes “P” and “D” (FIGS. 3 and 4) are joined together. In one exemplary embodiment, which will be described in greater detail herein, the first carriage 14 is stationary with respect to the remainder of the housing 12, while the second carriage 16 is movable with respect to the first carriage 14.

The first carriage 14 includes an upper jaw 18 and a lower jaw 20, with the upper jaw 18 being movable between an open condition (FIGS. 2 and 3) and a closed condition (FIG. 4). The upper jaw 18 of the illustrated embodiment is pivotal between the open and closed conditions, but it should be understood that the upper jaw 18 may be otherwise movable between the open and closed conditions (e.g., via vertical translational movement) without departing from the scope of the present disclosure.

When the upper jaw 18 is in the open condition, the lower jaw 20 is exposed or uncovered, which allows a pair of tubes P and D to be mounted within the first carriage 14 (at the beginning of a sterile connection procedure) and allows for a joined tube “J” (FIGS. 12 and 13) to be removed from the first carriage 14 (at the end of a sterile connection procedure). The lower jaw 20 of the first carriage 14 defines a first portion 22a of a proximal slot 22 and a first portion 24a of a distal slot 24 that is parallel to the first portion 22a of the proximal slot 22 (FIG. 2). In the illustrated embodiment, the first portions 22a and 24a of the proximal and distal slots 22 and 24 are substantially identical, but it should be understood that they may be differently configured without departing from the scope of the present disclosure. Regardless of their exact configuration, each of the first portions 22a, 24a is sized and configured to accommodate a portion of a tube that is to be joined to the tube received by the other slot 22, 24. This may include each first portion 22a, 24a having a general arcuate or V-shaped profile to facilitate proper positioning (namely, centering) of a tube inserted into the slot 22, 24.

When the upper jaw 18 of the first carriage 14 is in its closed condition (FIG. 4), it covers or overlays tubes received within the slots 22 and 24 to retain the tubes in position during a sterile connection procedure. The illustrated upper jaw 18 includes a latch 26 that is configured to engage a pin 28 of the lower jaw 20 when the upper jaw 18 is in the closed condition. Such an arrangement prevents inadvertent movement of the upper jaw 18 from the closed condition to the open condition, though it should be understood that other locking arrangements (e.g., a magnetic interlock) may be employed without departing from the scope of the present disclosure.

The second carriage 16 is similarly configured to the first carriage 14, with an upper jaw 30 that may be configured in accordance with the foregoing description of the upper jaw 18 of the first carriage 14 and a lower jaw 32 that may be configured in accordance with the foregoing description of the lower jaw 20 of the first carriage 14.

As will be described in greater detail, the second carriage 16 may be configured to move with respect to the first carriage 14 (and with respect to the housing 12), but in an initial or default position, the second carriage 16 is positioned with a second portion 22b of the proximal slot 22 (which is defined by the lower jaw 32 of the second carriage 16) aligned with the first portion 22a of the proximal slot 22 and a second portion 24b of the distal slot 24 (which is defined by the lower jaw 32 of the second carriage 16) aligned with the first portion 24a of the distal slot 24. With the second carriage 16 in such an initial or default position (and the upper jaws 18 and 30 in their open conditions), a proximal tube P may be inserted into the proximal slot 22, with the proximal tube P being partially received by the first portion 22a of the proximal slot 22 (defined by the lower jaw 20 of the first carriage 14) and partially received by the second portion 22b of the proximal slot 22 (defined by the lower jaw 32 of the second carriage 16).

Similarly, a distal tube D may be inserted into the distal slot 24, with the proximal tube P being partially received by the first portion 24a of the distal slot 24 (defined by the lower jaw 20 of the first carriage 14) and partially received by the second portion 24b of the distal slot 24 (defined by the lower jaw 32 of the second carriage 16). A conventional sterile connection device may require each tube to be joined to have a length of at least 31 mm, but sterile connection devices according to the present disclosure may be capable of joining shorter tubes, which may include tubes having a length on the order of approximately 22 mm.

The proximal and distal tubes P and D are mounted to the sterile connection device 10 in opposing orientations, with an end of one of the tubes P, D positioned closer to the first carriage 14 and an end of the other one of the tubes P, D positioned closer to the second carriage 16. To that end, the upper surface or face of the housing 12 may be provided with indicia to indicate the positions and orientations into which the tubes P and D are to be placed at the beginning of a sterile connection procedure.

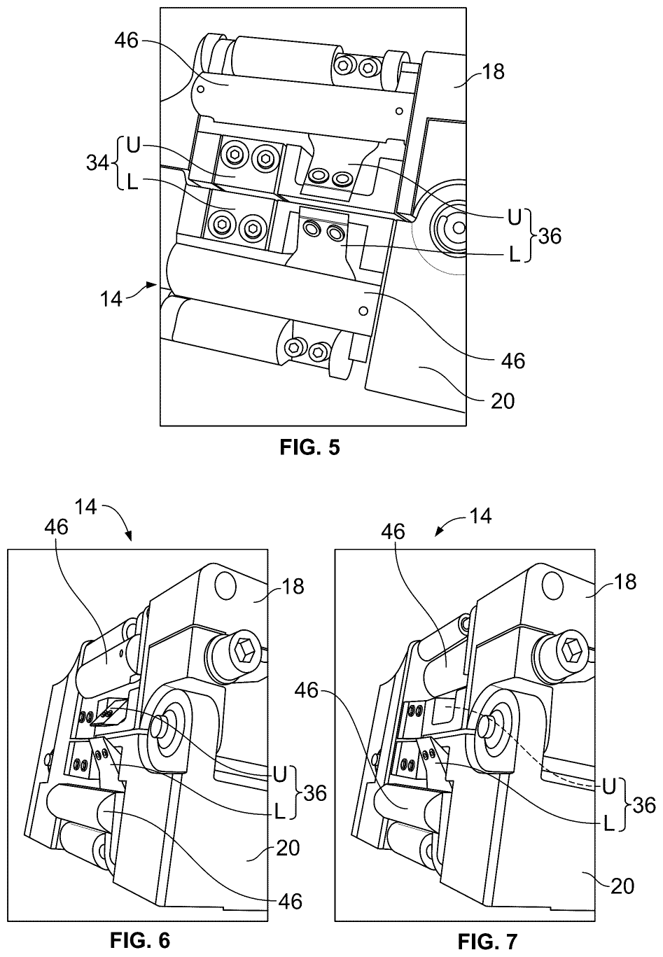

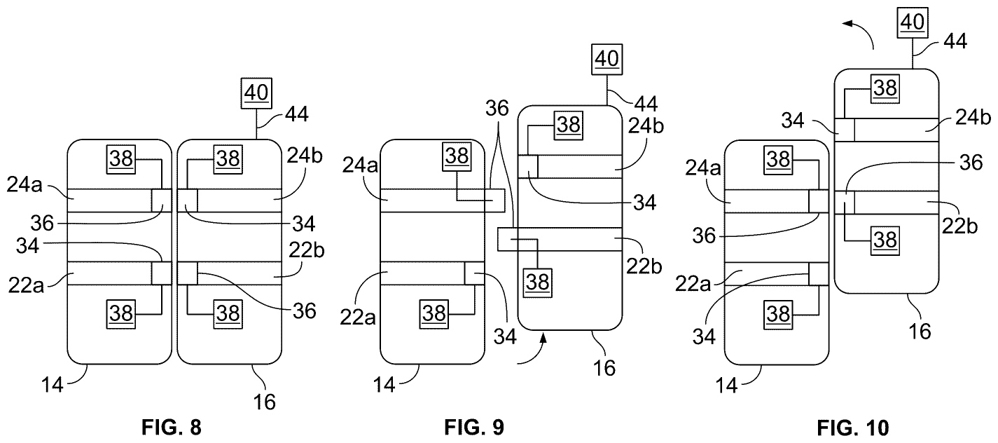

Each portion of each slot 22, 24 has a heating element associated with it, as best shown in FIGS. 5-10. One portion of each slot 22, 24 of each carriage 14, 16 has a waste-end heating element 34 associated with it, while the portion of the other slot 22, 24 defined by that carriage 14, 16 has a joining heating element 36 associated with it. The heating elements 34 and 36 are positioned at the side of each carriage 14, 16 facing the other carriage 14, 16, with each slot 22, 24 having one waste-end heating element 34 positioned adjacent to a joining heating element 36 when the carriages 14 and 16 are in their initial position (FIG. 8). The individual heating elements may be variously configured without departing from the scope of the present disclosure. However, in one exemplary embodiment, each heating element 34, 36 is configured as a pair of electrodes, with one upper electrode “U” associated with or incorporated into the upper jaw of a carriage and the other lower electrode “L” associated with or incorporated into the lower jaw of that same carriage (FIGS. 5-7). In such an embodiment, the electrodes may be formed of any of a variety of suitable materials, which may vary depending on the manner in which the tubes P and D are heated. For example, if radio frequency (“RF”) energy is employed to heat the tubes P and D, the electrodes may be formed of a metal (or combination of metals) with high electrical conductivity, such as stainless steel, brass, or titanium.

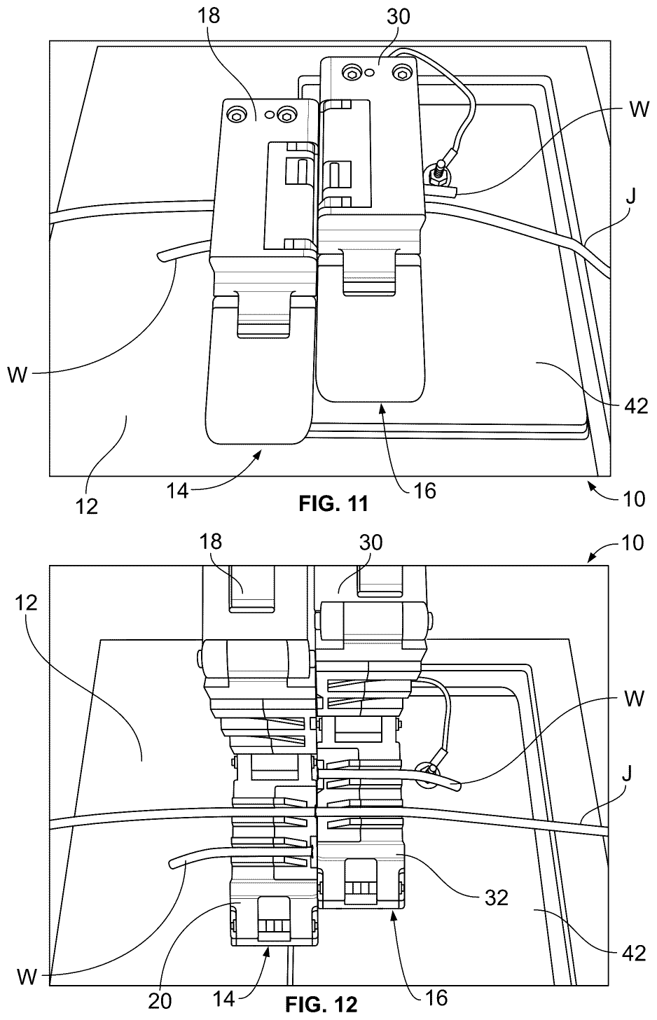

As will be described in greater detail, the waste-end heating elements 34 function to heat a portion of a tube in preparation for an end of the tube to be removed from the remainder of the tube as a waste product (hence the term “waste-end” being used to identify these heating elements). In the illustrated embodiment, the “waste-end” W of the proximal tube P is positioned adjacent to the first carriage 14, with the “waste-end” W of the distal tube D being positioned adjacent to the second carriage 16, as shown in FIGS. 11 and 12. Accordingly, the first portion 22a of the proximal slot 22 (which receives the “waste-end” W of the proximal tube P) is provided with a waste-end heating element 34, with the second portion 24b of the distal slot 24 (which receives the “waste-end” W of the distal tube D) being provided with a waste-end heating element 34, as best shown in FIGS. 8-10. FIG. 5 shows the side of the first carriage 14 that faces the second carriage 16, with the first portion 22a of the proximal slot 22 having a waste-end heating element 34 comprised of a lower electrode L incorporated into the lower jaw 20 and a corresponding upper electrode U being incorporated into the upper jaw 18. It should be understood that the second portion 24b of the distal slot 24 may similarly have a waste-end heating element 34 comprised of a lower electrode L incorporated into the lower jaw 32 of the second carriage 16 and an upper electrode U incorporated into the upper jaw 30 of the second carriage 16.

As noted above, each slot 22, 24 has one waste-end heating element 34 and one joining heating element 36. Thus, in the illustrated embodiment, a joining heating element 36 is associated with or incorporated into the second portion 22b of the proximal slot 22, while another joining heating element 36 is associated with or incorporated into the first portion 24a of the distal slot 24. FIGS. 5-7 show the first portion 24a of the distal slot 24 as having a joining heating element 36 comprised of a lower electrode L incorporated into the lower jaw 20 of the first carriage 14 and a corresponding upper electrode U being incorporated into the upper jaw 18. It should be understood that the second portion 22b of the proximal slot 22 may similarly have a joining heating element 36 comprised of a lower electrode L incorporated into the lower jaw 32 of the second carriage 16 and an upper electrode U incorporated into the upper jaw 30 of the second carriage 16. As will be described in greater detail, the joining heating elements 36 function to heat a portion of a tube P. D (particularly, the end of the tube remaining after the “waste-end” W has been removed) in preparation for the tubes P and D to be joined together (hence the term “joining” being used to identify these heating elements).

Each heating element 34, 36 is coupled to an energy source that is actuated to cause the heating element 34, 36 to heat the portion of the tube positioned adjacent to that heating element 34, 36. The nature of the energy sources may vary without departing from the scope of the present disclosure. However, according to one embodiment, each heating element 34, 36 is coupled to an energy source 38 (FIGS. 8-10) configured as a source of RF energy, which is actuated to cause the heating element 34, 36 to heat the associated tube P, D (without the heating element itself being heated). In such an embodiment, only RF energy is used to sever and subsequently join the tubes P and D, which may be advantageous compared to conventional devices requiring multiple forms of energy (e.g., RF energy and radiant heat) to sever and join a pair of tubes, due to its relative simplicity. In other embodiments, the energy sources of the waste-end heating elements 34 may be different from the energy sources of the joining heating elements 36. For example, one type of heating element (either the waste-end heating elements 34 or the joining heating elements 36) may be associated with an RF energy source, while the other type of heating elements may be associated with an energy source including a laser (in which case the tubes P and D may be heated by laser heating) or an energy source configured to cause the associated heating elements to apply radiant heat to the tubes P and D.

As described above, one or both of the carriages 14 and 16 is movable with respect to the other carriage 14, 16 (and with respect to the housing 12 of the sterile connection device 10). While it is within the scope of the present disclosure for both carriages 14 and 16 to be movable, only the second carriage 16 is movable in an exemplary embodiment. In such an embodiment, a motor 40 (FIGS. 8-10) is configured to be actuated to cause movement of the second carriage 16 with respect to the first carriage 14 (and with respect to the housing 12). While actuation of the motor 40 will be described herein as moving the second carriage 16, it should be understood that the motor 40 may be configured to move a component of the sterile connection device 10 secured to or otherwise associated with the second carriage 16, with the second carriage 16 being moved along with that other component. For example, in the illustrated embodiment, the second carriage 16 is mounted onto a plate or platform 42 that serves as a bi-directional parallel guide, facilitating free translational movement in the plane of the plate or platform 42, while preventing changes in the angular orientation of the second carriage 16.

In any event, the motor 40 moves the second carriage 16 from an initial position (FIG. 8), through one or more intermediate positions (FIG. 9), and to a final position (FIG. 10). FIGS. 8-10 illustrate the second carriage 16 moving through a substantially semicircular arcuate path with movement of the second carriage 16 from the initial position (FIG. 8) to the intermediate position (FIG. 9) being along a first portion of the arcuate path and movement of the second carriage 16 from the intermediate position to the final position (FIG. 10) being along a second portion of the arcuate path. In such an embodiment, the second carriage 16 may be coupled to the motor 40 (either directly or indirectly) by an eccentric shaft 44 or the like that is configured to convert operation of the motor 40 into movement of the second carriage 16 through an arcuate path. Regardless of the particular linkage between the motor 40 and the second carriage 16, the motor 40 may be configured to be operated in a first or forward direction to move the second carriage 16 from the initial position to the final position, with the motor 40 being operated in a second, opposite or reverse direction to move the second carriage 16 from the final position back to the initial position. In an alternative embodiment, the drive assembly may instead be configured such that operation of the motor 40 in a single direction alternately causes movement of the second carriage 16 between the initial and final positions.

FIGS. 8-10 illustrate movement of the second carriage 16 from the initial position to the intermediate position as being along an approximately 90° arc, with movement of the second carriage 16 from the intermediate position to the final position as being along a subsequent 90° arc (to complete 180° of travel along the substantially semicircular path). However, it should be understood that the portions of the path traversed by the second carriage 16 in moving from the initial position to the intermediate position and from the intermediate position to the final position may be different. For example, the motor 40 may be actuated to move the second carriage 16 through a 30° arc, followed by the motor 40 being actuated a second time to move the second carriage 16 through a 150° arc from the intermediate position to the final position. As will be explained in greater detail, the joining heating elements 36 are configured to heat severed ends of the tubes P and D when the second carriage 16 is in the intermediate position, so an intermediate position that is sufficiently spaced from the initial position to ensure that the “waste-ends” W of the tubes P and D have been removed by the time that the second carriage 16 has reached the intermediate position may be advantageous.

While FIGS. 8-10 illustrate a substantially semicircular arcuate path, it should be understood that the second carriage 16 may be moved through any path from the initial position to the final position without departing from the scope of the present disclosure. This may include the second carriage 16 being moved along a relatively simple path (e.g., a linear path or a non-semicircular curving path) or a more complex or irregular path. A relatively simple path may be advantageous (compared to a more complex path) to the extent that a single motor may be sufficient to control movement of the second carriage 16 through the entire path. On the other hand, a more complex or irregular path (which may require a plurality of motors to control movement of the second carriage 16 along the path) may be advantageous or even necessary in some cases (e.g., if the second carriage 16 must be moved in a first direction by a first motor to implement one stage of a sterile connection procedure and moved in a second direction by a second motor to implement another stage of the procedure).

As noted above, the sterile connection device 10 may include a controller, which may be variously configured without departing from the scope of the present disclosure, provided that it is configured to coordinate the various tasks carried out by the components of the sterile connection device 10 during a sterile connection procedure. In one embodiment, the controller may include a microprocessor (which, in fact may include multiple physical and/or virtual processors). According to other embodiments, the controller may include one or more electrical circuits designed to carry out the actions described herein. In fact, the controller may include a microprocessor and other circuits or circuitry. In addition, the controller may include one or more memories. The instructions by which the microprocessor is programmed may be stored on the memory associated with the microprocessor, which memory/memories may include one or more tangible non-transitory computer readable memories, having computer executable instructions stored thereon, which when executed by the microprocessor, may cause the microprocessor to carry out one or more actions as described herein.

Turning now to an exemplary sterile connection procedure, with the upper jaws 18 and 30 in their open condition (as in FIG. 2) an operator places two tubes P and D into the proximal and distal slots 22 and 24 defined by the lower jaws 20 and 32 of the carriages 14 and 16. The end of the proximal tube P (which will become the “waste-end” W) is positioned adjacent to the first carriage 14, while the end of the distal tube D (which will become the “waste-end” W) is positioned adjacent to the second carriage 16, as shown in FIG. 3. It should be understood that this orientation of the tubes P and D is merely exemplary and that the orientation may be reversed (with the “waste-end” W of the proximal tube P positioned adjacent to the second carriage 16 and the “waste-end” W of the distal tube D positioned adjacent to the first carriage 14), provided that the heating elements 34 and 36 are properly positioned (i.e., with the waste-end heating elements 34 associated with the portions of the slots 22 and 24 configured to receive the “waste-ends” W of the tubes P and D).

With the tubes P and D in place, the operator moves the upper jaws 18 and 30 from their open condition to their closed condition (FIG. 4). The upper jaws 18 and 30 may be independently movable between their open and closed conditions or may be configured to move together from the open condition to the closed condition and/or from the closed condition to the open condition. As described above, in their closed conditions, the upper jaws 18 and 30 cover the lower jaws 20 and 32 and secure the tubes P and D within the carriages 14 and 16.

With the upper jaws 18 and 30 in their closed condition, the operator presses a “start” button to continue the sterile connection procedure. Pressing the “start” button causes the controller to actuate the energy sources 38 coupled to the waste-end heating elements 34, which causes the waste-end heating elements 34 to heat the tubes P and D to a desired temperature. The exact temperature to which the tubes P and D are heated by the waste-end heating elements 34 may vary without departing from the scope of the present disclosure. In one embodiment, the tubes P and D are heated to a temperature that is less than the melting temperature of the material of which the tubes P and D are formed. A similar effect may be achieved by heating the tubes P and D to a temperature that is equal to or greater than the melting temperature and then allowing the tubes P and D to cool until they reach a temperature that is less than the melting temperature. In either case, such an approach prevents the tubes P and D from being melted by the waste-end heating elements 34, which prevents the formation of threads or fibers when the “waste-ends” W of tubes P and D are subsequently removed (as will be described).

Upon the tubes P and D reaching the target temperature (e.g., as determined by the controller, based upon a signal received from thermocouples or the like), the motor 40 is actuated to move the second carriage 16 away from its initial position (FIGS. 4 and 8) and into its intermediate position (FIG. 9). The waste-end heating elements 34 may be deactivated at any appropriate time, such as upon the tubes P and D reaching the target temperature or upon the motor 40 being actuated to move the second carriage 16 away from its initial position. In one embodiment, the waste-end heating elements 34 are configured to continue heating the tubes P and D after the second carriage 16 has been moved away from its initial position.

The tubes P and D are secured within the carriages 14 and 16, such that movement of the second carriage 16 away from the first carriage 14 applies tension to the tubes P and D. Continued movement of the second carriage 16 away from its initial position and toward its intermediate position applies increasing tension to the tubes P and D until they are each pulled apart or severed at the sections heated by the waste-end heating elements 34, thus removing the “waste-ends” W of tubes P and D and leaving the body of each tube P. D with an exposed severed end. As the tubes P and D are “cold” (i.e., not molten) at the points at which they are torn apart, threads or fibers are not formed when the “waste-ends” W are removed and, thus, will not remain when the severed ends of the tubes P and D are later joined (compare the conventional joint of FIG. 1 to a joint formed according to the present disclosure, which is shown in FIG. 13).

In addition to applying tension to the tubes P and D, movement of the second carriage 16 away from the first carriage 14 also causes each joining heating element 36 to move from a retracted condition (FIGS. 7 and 8) to a deployed condition (FIGS. 5, 6, and 9). The mechanism by which the joining heating elements 36 move from their retracted condition to their deployed condition may vary without departing from the scope of the present disclosure. In an exemplary embodiment, the movement is automatic and passive, not requiring actuation of a motor or other electromechanical driving force. In the illustrated embodiment, each joining heating element 36 includes an associated bumper 46 (FIGS. 5-7), which is configured to move between the retracted and deployed conditions with the associated joining heating element 36. When each joining heating element 36 comprises a pair of electrodes U and L (as in the illustrated embodiment), each electrode U, L of the joining heating element 36 may have its own associated bumper 46.

Each bumper 46 is biased to the deployed condition (e.g., by an associated spring or similar resilient element), but positioned such that the bumper 46 contacts the opposing carriage 14, 16 when the second carriage 16 is in the initial and final positions so as to retain the bumper 46 (and associated joining heating element 36) in its retracted condition. Accordingly, when the second carriage 16 is moved away from its initial position (and away from the first carriage 14), the bumpers 46 will begin to automatically and passively move from their retracted condition to their deployed condition. The bumpers 46 and their associated biasing mechanisms are configured such that the bumpers 46 and joining heating elements 36 have moved into their deployed conditions by the time that the second carriage 16 reaches its intermediate position (FIG. 9).

When the second carriage 16 reaches its intermediate position, the motor 40 is temporarily deactivated to retain the second carriage 16 in the intermediate position. The joining heating elements 36 (which have moved into their deployed conditions) are actuated for at least a portion of the time that the second carriage 16 is in its intermediate position to heat the severed ends of the tubes P and D to a second target temperature (e.g., as determined by the controller, based upon a signal received from thermocouples or the like). The joining heating elements 36 may be actuated before the second carriage 16 reaches its intermediate position and may remain in operation until some time after the second carriage 16 has moved away from the intermediate position and toward its final position. In any event, the joining heating elements 36 are configured and actuated to heat the severed ends of the tubes P and D to a temperature that is greater than or equal to the melting temperature of the tube material so as to cause the severed ends to melt or become molten. Thus, it will be seen that the joining heating elements 36 operate to heat the tubes P and D to a temperature that is greater than the temperature of the tubes P and D at the time they are severed (after being heated by the waste-end heating elements 34).

In the interest of avoiding electric arcs (which may be possible if heating a liquid-filled tube via RF energy), any of a number of possible safeguards may be employed. For example, RF current may be monitored and controlled so as to avoid electric arcs. According to another approach, the joining heating elements 36 may be configured to avoid local field intensity maxima and/or be galvanically insulated. According to yet another approach, steps may be taken to move any liquid in the tubes P and D away from the joining heating elements 36 (when active). In another embodiment, if application of RF energy is to be avoided, radiant heat may instead be employed by the joining heating elements 36 to heat the tubes P and D (e.g., with retracting electrodes being replaced by retracting heat shields).

As noted above, it is within the scope of the present disclosure for the waste-end heating elements 34 to continue operating after the second carriage 16 has been moved away from its initial position. This may include the waste-end heating elements 34 operating to heat the “waste-ends” W of the tubes P and D while the second carriage 16 is in its intermediate position, which may be advantageous to ensure that the “waste-ends” W are safely sealed before the end of the procedure. In such an embodiment, operation of the waste-end heating elements 34 and the joining heating elements 36 may overlap (with the joining heating elements 36 beginning to heat the tubes P and D before the waste-end heating elements 34 are deactivated) or the joining heating elements 36 may remain inactive until the waste-end heating elements 34 are deactivated (at which time the joining heating elements 36 may be actuated).

When the controller has determined that the severed ends of the tubes P and D have been heated to an appropriate temperature, it commands the motor 40 to complete the movement of the second carriage 16 by bringing it from its intermediate position (FIG. 9) to its final position (FIGS. 10 and 11). The joining heating elements 36 are configured to return to their retracted condition by the time that the second carriage 16 reaches its final position so as to expose the (heated/molten) severed ends of the tubes P and D. The mechanism by which the joining heating elements 36 are moved from their deployed condition back to their retracted condition may vary without departing from the scope of the present disclosure. However, in the illustrated embodiment, movement of the second carriage 16 from its intermediate position to its final position brings the bumpers 46 associated with the joining heating elements 36 into contact with the opposing carriage 14, 16, thus automatically and passively pressing the bumpers 46 (and, hence, the associated joining heating elements 36) back to their retracted condition.

With the second carriage 16 in its final position and the joining heating elements 36 in their retracted condition, the severed ends of the tubes P and D will be exposed, aligned, and moved into contact with each other. The severed ends of the tubes P and D are pressed together for a predetermined amount of time (e.g., seven seconds) to create a joint and cool. In one embodiment, the waste-end heating elements 34 may be activated again with the second carriage 16 in its final position in order to heat the “waste-ends” and ensure that they are safely sealed.

When joint has been created and allowed to cool, the controller advances to the next stage of the procedure. In this stage, the operator is notified that the tubes P and D have been sterilely connected to define a joined tube J (FIG. 11). This notification may be provided in the form of an audible alert (e.g., an alarm) and/or a visual alert (e.g., a flashing light or an icon shown on a screen), for example. At this time, the controller unlocks the upper jaws 18 and 30 (if they are locked into their closed condition), which allows the operator to move the upper jaws 18 and 30 back into their open condition (FIG. 12) and then remove the joined tube J from the lower jaws 20 and 32 of the carriages 14 and 16 (FIG. 13), along with the “waste-ends” W of the tubes P and D, which may be discarded. The joined tube J is subsequently manipulated (either manually or by a suitably configured device) to open the joint for fluid flow (e.g., by pinching the joint) and ensure that the joint is secure.

At the end of the procedure, the operator may press a “reset” button to reset the sterile connection device 10, which may include the controller commanding the motor 40 to return the second carriage 16 to its initial position.

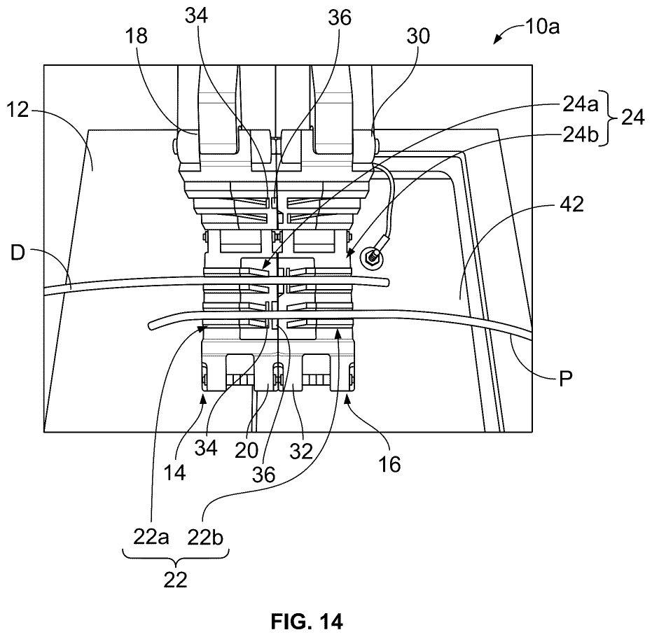

FIGS. 14-18 illustrate another exemplary embodiment of a sterile connection device 10a according to the present disclosure. The sterile connection device 10a of FIGS. 14-18 is similarly configured to the sterile connection device 10 of FIGS. 2-12 (with similar components being correspondingly labeled and being configured as described above, except where noted to the contrary), with two carriages 14 and 16 (at least one being movable), four heating elements 34 and 36, and multiple heating phases. However, rather than all four portions of the slots 22 and 24 including one heating element, the sterile connection device 10a of FIGS. 14-18 instead has two portions 22b and 24a with no heating element. As for the other two portions 22a and 24b, each has a waste-end heating element 34 and a joining heating element 36. In accordance with the above description of the embodiment of FIGS. 14-18, each heating element may be variously configured without departing from the scope of the present disclosure, which may include each heating element 34, 36 comprising an upper electrode and a lower electrode or an upper heat shield and a lower heat shield (as shown in FIG. 14).

As for the manner in which the sterile connection device 10a of FIG. 14 is used to execute a sterile connection procedure, it is similar to the above-described procedure, with some notable differences. The procedures begin similarly, with an operator placing two tubes P and D into the proximal and distal slots 22 and 24 defined by the lower jaws 20 and 32 of the carriages 14 and 16 (as shown in FIG. 14). As in the above-described procedure, the end of the proximal tube P (which will become the “waste-end” W) is positioned adjacent to the first carriage 14, while the end of the distal tube D (which will become the “waste-end” W) is positioned adjacent to the second carriage 16 (i.e., in the portions 22a of the slots 24b having the heating elements 34 and 36).

With the tubes P and D in place, the operator moves the upper jaws 18 and 30 from their open condition to their closed condition to secure the tubes P and D within the carriages 14 and 16 (as described above). The operator presses the “start” button to cause the controller to actuate the energy sources (not illustrated) coupled to the waste-end heating elements 34, which causes the waste-end heating elements 34 to heat the tubes P and D to a desired temperature. As described above, the waste-end heating elements 34 may heat the tubes P and D to a temperature that is less than the melting temperature of the material of which the tubes P and D are formed or to a greater temperature, followed by a cooling phase in which the tubes P and D are allowed to cool until they reach a temperature that is less than the melting temperature.

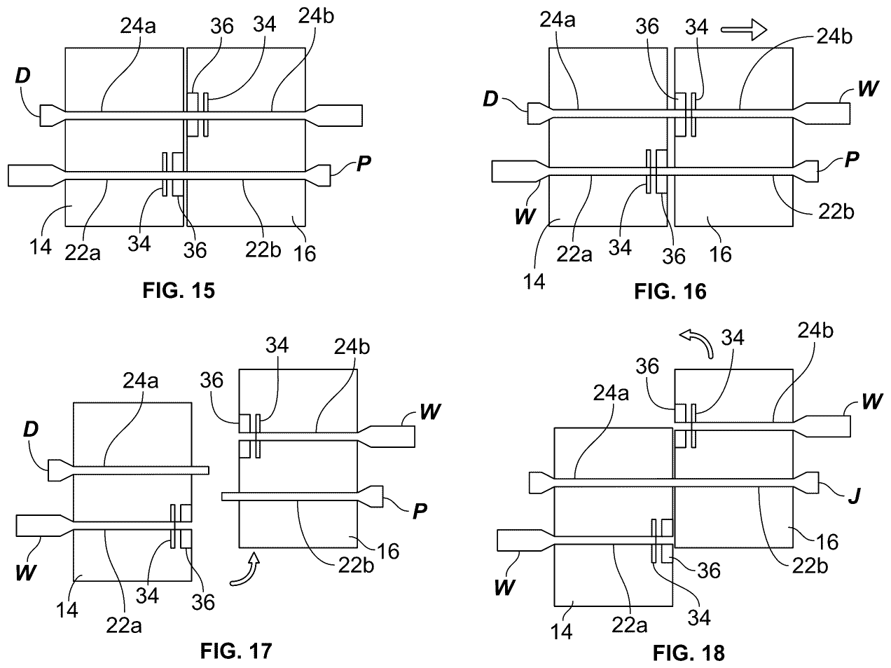

Upon the tubes P and D reaching the target temperature, the motor is actuated to move the second carriage 16 away from its initial position (FIG. 15) and into an intermediate position (FIG. 16). The waste-end heating elements 34 may be deactivated at any appropriate time, such as upon the tubes P and D reaching the target temperature or upon the motor being actuated to move the second carriage 16 away from its initial position.

The tubes P and D are secured within the carriages 14 and 16, such that movement of the second carriage 16 away from the first carriage 14 applies tension to the tubes P and D. Continued movement of the second carriage 16 away from its initial position and toward its intermediate position applies increasing tension to the tubes P and D until they are each pulled apart or severed at the sections heated by the waste-end heating elements 34, thus removing the “waste-ends” W of tubes P and D and leaving the body of each tube P. D with an exposed severed end.

When the second carriage 16 reaches its intermediate position, the motor is temporarily deactivated to retain the second carriage 16 in the intermediate position. The joining heating elements 36 are actuated for at least a portion of the time that the second carriage 16 is in its intermediate position to heat the severed ends of the tubes P and D to a second target temperature (e.g., as determined by the controller, based upon a signal received from thermocouples or the like) that is greater than or equal to the melting temperature of the tube material so as to cause the severed ends to melt or become molten.

It will be seen that the intermediate position of FIG. 16 is closer to the initial position (FIG. 15) than in the above-described procedure, which is due to each joining heating element 36 being incorporated into the same carriage as the associated waste-end heating element 34. As such, the second carriage 16 is moved only a small distance from its initial position to its intermediate position to properly position the severed ends of the tubes P and D with respect to the joining heating elements 36. It is within the scope of the present disclosure for the sterile connection device 10 of FIGS. 2-12 to be moved into an intermediate position that is similar to the one shown in FIG. 16, though a greater separation between the carriages 14 and 16 may be advantageous to ensure that the joining heating elements 36 move from their retracted condition to their deployed condition.

Notably, the joining heating elements 36 of the embodiment of FIGS. 14-18 are not movable between retracted and deployed conditions, but rather are static and secured in place within the carriages 14 and 16, such that each joining heating element 36 remains positioned adjacent to the associated waste-end heating element 34 throughout an entire sterile connection procedure. As the joining heating elements 36 are not retractable, but remain in a deployed condition, the carriages 14 and 16 may remain relatively close together when the joining heating elements 36 are actuated to heat the severed ends of the tubes P and D. Compared to the embodiment of FIGS. 2-12, the embodiment of FIGS. 14-18 has fewer moving parts, which may make it especially robust and inexpensive.

When the controller has determined that the severed ends of the tubes P and D have been heated to an appropriate temperature, it commands the motor to complete the movement of the second carriage 16 by bringing it from its intermediate position (FIG. 16) to its final position (FIG. 18). FIGS. 15-18 illustrate the second carriage 16 being moved through an arcuate or substantially semi-circular path (with FIG. 17 illustrating a second intermediate position between the intermediate position of FIG. 16 and the final position of FIG. 18), in which case the movement of the second carriage 16 from its initial position (FIG. 15) to the intermediate position of FIG. 16 may be a small segment of such an arcuate path. However, as in the above-described procedure, it is within the scope of the present disclosure for the second carriage 16 of the sterile connection device 10a to move through a non-arcuate path from its initial position to its final position.

With the second carriage 16 in its final position (FIG. 18), the severed ends of the tubes P and D will be aligned and moved into contact with each other. The severed ends of the tubes P and D are pressed together for a predetermined amount of time (e.g., seven seconds) to create a joint and cool. In one embodiment, the waste-end heating elements 34 may be activated again with the second carriage 16 in its final position in order to heat the “waste-ends” W and ensure that they are safely sealed.

When joint has been created and allowed to cool, the controller advances to the next stage of the procedure. In this stage, the operator is notified that the tubes P and D have been sterilely connected to define a joined tube J. At this time, the controller unlocks the upper jaws 18 and 30 (if they are locked into their closed condition), which allows the operator to move the upper jaws 18 and 30 back into their open condition and then remove the joined tube J from the lower jaws 20 and 32 of the carriages 14 and 16, along with the “waste-ends” W of the tubes P and D, which may be discarded. The joined tube J is subsequently manipulated (either manually or by a suitably configured device) to open the joint for fluid flow (e.g., by pinching the joint) and ensure that the joint is secure.

At the end of the procedure, the operator may press a “reset” button to reset the sterile connection device 10, which may include the controller commanding the motor to return the second carriage 16 to its initial position.

It should again be emphasized that the illustrated sterile connection devices 10 and 10a are merely exemplary and that sterile connection devices according to the present disclosure may be differently configured without departing from the scope of the present disclosure. This may include a sterile connection device having its components differently arranged and/or a sterile connection device including additional components (e.g., a cord for connection to an external power source, a variety of sensors, and/or a touchscreen for use by an operator).

### Aspects

Aspect 1. A sterile connection device comprising: a first carriage including a first lower jaw defining a first portion of a proximal slot configured to receive a portion of a proximal tube and defining a first portion of a distal slot configured to receive a portion of a distal tube, and a first upper jaw configured to move between an open condition spaced away from the first lower jaw and a closed condition positioned adjacent to the first lower jaw; a second carriage positioned laterally of the first carriage and including a second lower jaw defining a second portion of the proximal slot and a second portion of the distal slot, and a second upper jaw configured to move between an open condition spaced away from the second lower jaw and a closed condition positioned adjacent to the second lower jaw; and a motor configured to move the second carriage from an initial position to a final position, wherein the first and second portions of the proximal slot are aligned in the initial position, the first and second portions of the distal slot are aligned in the initial position, one of the portions of the proximal slot is aligned with one of the portions of the distal slot in the final position, and the sterile connection device includes no other motors.
Aspect 2. The sterile connection device of Aspect 1, wherein the motor is configured to move the second carriage through an arcuate path from the initial position to the final position.
Aspect 3. The sterile connection device of Aspect 2, wherein the motor is configured to move the second carriage through a first portion of the arcuate path from the initial position to an intermediate position, pause movement of the second carriage in the intermediate position, and move the second carriage through a second portion of the arcuate path from the intermediate position to the final position.
Aspect 4. The sterile connection device of Aspect 3, wherein the tubes are heated when the second carriage is in the initial and intermediate positions.
Aspect 5. The sterile connection device of Aspect 4, wherein the tubes are heated via application of only radio frequency energy.
Aspect 6. The sterile connection device of any one of Aspects 4-5, wherein each tube is heated to a first temperature when the second carriage is moved away from the initial position, and each tube is heated to a second temperature greater than the first temperature when the second carriage is in the intermediate position.
Aspect 7. The sterile connection device of Aspect 6, wherein the first temperature is configured to be less than a melting temperature of the tubes, and the second temperature is configured to be greater than or equal to the melting temperature of the tubes.
Aspect 8. The sterile connection device of any one of the preceding Aspects, wherein each carriage includes a waste-end heating element associated with one of the portions of the slots defined by said carriage and a joining heating element associated with said one of the portions of the slots defined by said carriage or with the portion of the other slot defined by said carriage.
Aspect 9. The sterile connection device of Aspect 8, wherein each waste-end heating element is configured to be operative when the second carriage is in the initial position, and each joining heating element is configured to be operative when the second carriage is in neither the initial position nor the final position.
Aspect 10. The sterile connection device of any one of Aspects 8-9, wherein each joining heating element is configured to be movable with respect to the carriage with which said joining heating element is associated between a retracted condition and a deployed condition.
Aspect 11. The sterile connection device of Aspect 10, wherein each joining heating element is configured to be in the retracted condition when the second carriage is in the initial and final positions and in the deployed condition when said joining heating element is operative.
Aspect 12. The sterile connection device of any one of Aspects 10-11, wherein each joining heating element is configured to automatically move from the retracted condition toward the deployed condition when the second carriage is moved away from the initial position.
Aspect 13. The sterile connection device of any one of Aspects 10-12, wherein each joining heating element is configured to automatically move from the deployed condition to the retracted condition when the second carriage is moved into the final position.
Aspect 14. The sterile connection device of any one of Aspects 10-13, wherein each joining heating element is associated with a side of the associated carriage facing the other carriage, each joining heating element includes an associated bumper configured to move between the retracted and deployed conditions with the joining heating element, each bumper is biased to the deployed condition, and each bumper contacts the carriage with which said bumper is not associated when the second carriage is in the initial and final positions so as to retain said bumper in the retracted condition.
Aspect 15. The sterile connection device of any one of Aspects 8-14, wherein each heating element comprises a pair of electrodes.
Aspect 16. A sterile connection device comprising: a first carriage including a first lower jaw defining a first portion of a proximal slot configured to receive a portion of a proximal tube and defining a first portion of a distal slot configured to receive a portion of a distal tube, and a first upper jaw configured to move between an open condition spaced away from the first lower jaw and a closed condition positioned adjacent to the first lower jaw; a second carriage positioned laterally of the first carriage and including a second lower jaw defining a second portion of the proximal slot and a second portion of the distal slot, and a second upper jaw configured to move between an open condition spaced away from the second lower jaw and a closed condition positioned adjacent to the second lower jaw; and a motor configured to move at least one of the carriages from an initial position to a final position, wherein the first and second portions of the proximal slot are aligned in the initial position, the first and second portions of the distal slot are aligned in the initial position, and one of the portions of the proximal slot is aligned with one of the portions of the distal slot in the final position, each carriage includes a waste-end heating element associated with one of the portions of the slots defined by said carriage and a joining heating element associated with said one of the portions of the slots defined by said carriage or with the portion of the other slot defined by said carriage, each waste-end heating element is configured to be operative in the initial position, and each joining heating element is configured to be operative when said at least one of the carriages is in neither the initial position nor the final position.
Aspect 17. The sterile connection device of Aspect 16, wherein each joining heating element is configured to be movable with respect to the carriage with which said joining heating element is associated between a retracted condition and a deployed condition.
Aspect 18. The sterile connection device of Aspect 17, wherein each joining heating element is configured to be in the retracted condition when said at least one of the carriages is in the initial and final positions and in the deployed condition when said joining heating element is operative.
Aspect 19. The sterile connection device of any one of Aspects 17-18, wherein each joining heating element is configured to automatically move from the retracted condition toward the deployed condition when said at least one of the carriages is moved away from the initial position.
Aspect 20. The sterile connection device of any one of Aspects 17-19, wherein each joining heating element is configured to automatically move from the deployed condition to the retracted condition when said at least one of the carriages is moved into the final position.
Aspect 21. The sterile connection device of any one of Aspects 17-20, wherein each joining heating element is associated with a side of the associated carriage facing the other carriage, each joining heating element includes an associated bumper configured to move between the retracted and deployed conditions with the joining heating element, each bumper is biased to the deployed condition, and each bumper contacts the carriage with which said bumper is not associated when said at least one of the carriages is in the initial and final positions so as to retain said bumper in the retracted condition.
Aspect 22. The sterile connection device of Aspect 16, wherein each joining heating element is associated with the same portion of the slot as the associated waste-end heating element, and neither joining heating element is configured to be movable between a retracted condition and a deployed condition.
Aspect 23. The sterile connection device of Aspect 22, wherein each joining heating element is positioned laterally of the associated waste-end heating element.
Aspect 24. The sterile connection device of any one of Aspects 22-23, wherein each joining heating element is associated with a side of the associated carriage facing the other carriage.
Aspect 25. The sterile connection device of any one of Aspects 22-24, wherein each waste-end heating element and the associated joining heating element are positioned between the associated portion of the slot and a side of the associated carriage facing the other carriage.
Aspect 26. The sterile connection device of any one of Aspects 22-25, wherein the waste-end heating element and the joining heating element of one of the carriages are associated with the portion of the proximal slot defined by said one of the carriages, and the waste-end heating element and the joining heating element of the other one of the carriages are associated with the portion of the distal slot defined by said other one of the carriages.
Aspect 27. The sterile connection device of any one of Aspects 21-26, wherein the motor is configured to move the second carriage from the initial position to the final position.
Aspect 28. The sterile connection device of Aspect 27, wherein the motor is configured to move the second carriage through an arcuate path from the initial position to the final position.
Aspect 29. The sterile connection device of Aspect 28, wherein the motor is configured to move the second carriage through a first portion of the arcuate path from the initial position to an intermediate position, pause movement of the second carriage in the intermediate position, and move the second carriage through a second portion of the arcuate path from the intermediate position to the final position.
Aspect 30. The sterile connection device of Aspect 29, wherein each joining heating element is configured to be operative when the second carriage is in the intermediate position.
Aspect 31. The sterile connection device of any one of Aspects 21-30, wherein each heating element is configured to heat a portion of one of said tubes via application of radio frequency energy.
Aspect 32. The sterile connection device of any one of Aspects 21-31, wherein each waste-end heating element is configured to heat a portion of an associated tube to a first temperature when said at least one of the carriages is moved away from the initial position, and each joining heating element is configured to heat a portion of an associated tube to a second temperature greater than the first temperature when said joining heating element is operative.
Aspect 33. The sterile connection device of Aspect 32, wherein the first temperature is configured to be less than a melting temperature of the portion of the tube being heated by the waste-end heating element, and the second temperature is configured to be greater than or equal to the melting temperature of the portion of the tube being heated by the joining heating element.
Aspect 34. The sterile connection device of any one of Aspects 21-33, wherein each heating element comprises a pair of electrodes.
Aspect 35. The sterile connection device of any one of Aspects 21-34, wherein the sterile connection device includes no other motors.
Aspect 36. A sterile connection device comprising: a first carriage including a first lower jaw defining a first portion of a proximal slot configured to receive a portion of a proximal tube and defining a first portion of a distal slot configured to receive a portion of a distal tube, and a first upper jaw configured to move between an open condition spaced away from the first lower jaw and a closed condition positioned adjacent to the first lower jaw; a second carriage positioned laterally of the first carriage and including a second lower jaw defining a second portion of the proximal slot and a second portion of the distal slot, and a second upper jaw configured to move between an open condition spaced away from the second lower jaw and a closed condition positioned adjacent to the second lower jaw; and a motor configured to move at least one of the carriages from an initial position to an intermediate position to a final position, wherein in the initial position, the first and second portions of the proximal slot are aligned, the first and second portions of the distal slot are aligned, and a portion of each tube is heated, in moving said at least one of the carriages from the initial position to the intermediate position, the heated portion of each tube is torn to define a torn end, with the heated portion of each tube being torn while at a first temperature less than a melting temperature of said heated portion, in the intermediate position, the torn end of each tube is heated to a second temperature greater than or equal to a melting temperature of said torn end, and in the final position, one of the portions of the proximal slot is aligned with one of the portions of the distal slot so as to align and join the torn ends of the tubes.
Aspect 37. The sterile connection device of Aspect 36, wherein the sterile connection device includes no other motors.
Aspect 38. The sterile connection device of any one of Aspects 36-37, wherein the tubes are heated via application of only radio frequency energy.
Aspect 39. The sterile connection device of any one of Aspects 36-38, wherein each carriage includes a waste-end heating element associated with one of the portions of the slots defined by said carriage and a joining heating element associated with said one of the portions of the slots defined by said carriage or with the portion of the other slot defined by said carriage.
Aspect 40. The sterile connection device of Aspect 39, wherein each waste-end heating element is configured to be operative when said at least one of the carriages is in the initial position.
Aspect 41. The sterile connection device of any one of Aspects 39-40, wherein each joining heating element is configured to not be operative when said at least one of the carriages is in the initial and final positions.
Aspect 42. The sterile connection device of any one of Aspects 39-41, wherein each joining heating element is configured to be operative when said at least one of the carriages is in the intermediate position.
Aspect 43. The sterile connection device of any one of Aspects 39-42, wherein each joining heating element is configured to be movable with respect to the carriage with which said joining heating element is associated between a retracted condition and a deployed condition.
Aspect 44. The sterile connection device of Aspect 43, wherein each joining heating element is configured to be in the retracted condition when said at least one of the carriages is in the initial and final positions and in the deployed condition when said at least one of the carriages is in the intermediate position.
Aspect 45. The sterile connection device of any one of Aspects 43-44, wherein each joining heating element is configured to automatically move from the retracted condition toward the deployed condition when said at least one of the carriages is moved away from the initial position.
Aspect 46. The sterile connection device of any one of Aspects 43-45, wherein each joining heating element is configured to automatically move from the deployed condition to the retracted condition when said at least one of the carriages is moved into the final position.
Aspect 47. The sterile connection device of any one of Aspects 43-46, wherein each joining heating element is associated with a side of the associated carriage facing the other carriage, each joining heating element includes an associated bumper configured to move between the retracted and deployed conditions with the joining heating element, each bumper is biased to the deployed condition, and each bumper contacts the carriage with which said bumper is not associated when said at least one of the carriages is in the initial and final positions so as to retain said bumper in the retracted condition.
Aspect 48. The sterile connection device of any one of Aspects 39-47, wherein each heating element comprises a pair of electrodes.
Aspect 49. The sterile connection device of any one of Aspects 36-48, wherein the motor is configured to move the second carriage from the initial position to the intermediate and final positions.
Aspect 50. The sterile connection device of Aspect 49, wherein the motor is configured to move the second carriage through a first portion of an arcuate path from the initial position to the intermediate position, pause movement of the second carriage in the intermediate position, and move the second carriage through a second portion of the arcuate path from the intermediate position to the final position.
Aspect 51. A method of sterilely connecting two tubes, comprising: heating a portion of each of two tubes; severing each tube at the heated portion to define a severed end of each tube; moving the severed ends of the two tubes into contact with each other; and joining the severed ends of the tubes together, wherein the tubes are heated and joined without application of any type of energy other than radio frequency energy.
Aspect 52. The method of Aspect 52, wherein the tubes are heated and severed without cutting the tubes.
Aspect 53. The method of any one of Aspects 51-52, wherein said heated portions of the tubes are not molten when said heated portions of the tubes are severed.
Aspect 54. The method of any one of Aspects 51-53, further comprising heating the severed ends of the tubes before moving the severed ends of the tubes into contact with each other.
Aspect 55. The method of Aspect 54, wherein the severed ends of the tubes are heated by application of radio frequency energy and no other type of energy.
Aspect 56. The method of any one of Aspects 54-55, wherein the severed ends of the tubes are molten when the severed ends of the tubes are moved into contact with each other.
Aspect 57. The method of any one of Aspects 51-54, wherein the tubes are heated, severed, moved, and joined by operation of a single motor.
Aspect 58. The method of Aspect 57, further comprising mounting the two tubes within first and second carriages prior to heating the tubes, wherein the single motor is operated to move the second carriage from an initial position in which the tubes are heated to a final position in which the severed ends of the tubes are joined without moving the first carriage.
Aspect 59. The method of Aspect 58, wherein the second carriage is moved through an arcuate path when moving from the initial position to the final position.
Aspect 60. The method of Aspect 59, wherein the second carriage is moved through a first portion of the arcuate path from the initial position to an intermediate position, movement of the second carriage is paused at the intermediate position, and the second carriage is moved through a second portion of the arcuate path from the intermediate position to the final position.
Aspect 61. The method of Aspect 60, wherein the heated portions of the tubes are severed when the second carriage is moved from the initial position to the intermediate position.
Aspect 62. The method of any one of Aspects 60-61, wherein the severed ends of the tubes are heated when the second carriage is in the intermediate position.
Aspect 63. The method of Aspect 62, wherein each carriage includes a joining heating element configured to heat the severed end of a different one of the two tubes, each joining heating element is in a retracted condition when the second carriage is in the initial and final positions, and each joining heating element is in a deployed condition when the second carriage is in the intermediate position.
Aspect 64. The method of Aspect 63, wherein each joining heating element is configured to automatically move from the retracted condition toward the deployed condition when the second carriage is moved away from the initial position, and each joining heating element is configured to automatically move from the deployed condition to the retracted condition when the second carriage is moved into the final position.
Aspect 65. The method of any one of Aspects 63-64, wherein each joining heating element is associated with a side of the associated carriage facing the other carriage, each joining heating element includes an associated bumper configured to move between the retracted and deployed conditions with the joining heating element, each bumper is biased to the deployed condition, and each bumper contacts the carriage with which said bumper is not associated when the second carriage is in the initial and final positions so as to retain said bumper in the retracted condition.

It will be understood that the embodiments described above are illustrative of some of the applications of the principles of the present subject matter. Numerous modifications may be made by those skilled in the art without departing from the spirit and scope of the claimed subject matter, including those combinations of features that are individually disclosed or claimed herein. For these reasons, the scope hereof is not limited to the above description but is as set forth in the following claims, and it is understood that claims may be directed to the features hereof, including as combinations of features that are individually disclosed or claimed herein.

1-17. (canceled)

18. A method of sterilely connecting two tubes, comprising:
heating a portion of each of two tubes;
severing each tube at the heated portion to define a severed end of each tube;
moving the severed ends of the two tubes into contact with each other; and
joining the severed ends of the tubes together, wherein the tubes are heated and joined without application of any type of energy other than radio frequency energy.

19. The method of claim 18, wherein the tubes are heated and severed without cutting the tubes.

20. The method of claim 18, wherein said heated portions of the tubes are not molten when said heated portions of the tubes are severed.

21. The method of claim 18, further comprising heating the severed ends of the tubes before moving the severed ends of the tubes into contact with each other.

22. The method of claim 21, wherein the severed ends of the tubes are heated by application of radio frequency energy and no other type of energy.

23. The method of claim 21, wherein the severed ends of the tubes are molten when the severed ends of the tubes are moved into contact with each other.

24. The method of claim 18, wherein the tubes are heated, severed, moved, and joined by operation of a single motor.

25. The method of claim 24, further comprising mounting the two tubes within first and second carriages prior to heating the tubes, wherein the single motor is operated to move the second carriage from an initial position in which the tubes are heated to a final position in which the severed ends of the tubes are joined without moving the first carriage.

26. The method of claim 25, wherein the second carriage is moved through an arcuate path when moving from the initial position to the final position.

27. The method of claim 26, wherein
the second carriage is moved through a first portion of the arcuate path from the initial position to an intermediate position,
movement of the second carriage is paused at the intermediate position, and
the second carriage is moved through a second portion of the arcuate path from the intermediate position to the final position.

28. The method of claim 27, wherein the heated portions of the tubes are severed when the second carriage is moved from the initial position to the intermediate position.

29. The method of claim 27, wherein the severed ends of the tubes are heated when the second carriage is in the intermediate position.

30. The method of claim 29, wherein
each carriage includes a joining heating element configured to heat the severed end of a different one of the two tubes,
each joining heating element is in a retracted condition when the second carriage is in the initial and final positions, and
each joining heating element is in a deployed condition when the second carriage is in the intermediate position.

31. The method of claim 30, wherein
each joining heating element is configured to automatically move from the retracted condition toward the deployed condition when the second carriage is moved away from the initial position, and
each joining heating element is configured to automatically move from the deployed condition to the retracted condition when the second carriage is moved into the final position.

32. The method of claim 30, wherein
each joining heating element is associated with a side of the associated carriage facing the other carriage,
each joining heating element includes an associated bumper configured to move between the retracted and deployed conditions with the joining heating element,
each bumper is biased to the deployed condition, and
each bumper contacts the carriage with which said bumper is not associated when the second carriage is in the initial and final positions so as to retain said bumper in the retracted condition.
